(19) United States
(12) Patent Application Publication
(10) Pub. No.: US 2009/0111164 A1
(43) Pub. Date: Apr. 30, 2009

(54) METHOD AND PLANT FOR ANAEROBIC TREATMENT OF EFFLUENT CONTAINING CELLULAR MATERIALS

(76) Inventors: Paul Schall, Luchingen (DE); Ulf Theilen, Burgwedel (DE); Thomas Luthardt-Behle, Eberstadt (DE); Armin Gosch, Schomberg (DE); Susanne Veser, Biberach (DE); Christian Widmer, Binningen (CH)

Correspondence Address: BOYLE FREDRICKSON S.C., 840 North Plankinton Avenue, MILWAUKEE, WI, 53203, US

(21) Appl. No.: 11/997,293

(22) PCT Filed: Jul. 28, 2006
(86) PCT No.: PCT/DE2006/001312

§ 371 Date: Aug. 1, 2008

(30) Foreign Application Priority Data
Jul. 29, 2005 (DE) 102005036532.9

(51) Int. Cl. D06M 16/00; C12M 1/12

(52) U.S. Cl. 435/263; 435/290.1

(57) ABSTRACT

Disclosed are a method for the anaerobic treatment of pulp-containing waste and a fermentation plant for such products, wherein they are first of all mechanically prepared and comminuted, subsequently diluted to a predetermined dry matter content by the addition of process water or the like, and sanitized and anaerobically fermented in a subsequent method step. The remaining rotted residue is divided into a plastics-rich and a pulp-rich fraction, and these fractions are conditioned in conditioning stages to recyclable materials, or to fuel, or to products adapted to be deposited.

### BACKGROUND OF INVENTION

1. Field of the Invention

The invention relates to a method for the anaerobic treatment of pulp-containing waste, and to a fermentation plant suited for performing such a method.

2. Description of the Related Art

Pulp is, for instance, produced by a chemical disintegration of plant fibers, usually wood, and consists predominantly of cellulose. For the production of such cellulose, predominantly acid sulfite methods and alkaline sulfate methods are used. Recently, however, alternative methods such as the natural pulping method have also been used. Pulp is used in a plurality of products such as, for instance, cigarette filters, paper, paperboard containers, cotton batting and dressings, handkerchiefs, hygiene products, cellulose fibers for the reinforcement of cast or cement, or for the production of cellulose derivatives.

Although part of the waste paper accruing is subject to a preparation and can, for instance, be used for the manufacturing of paper, the predominant share of the paper/cardboard container waste is still incinerated. The operation of such waste incineration plants requires, however, considerable plant-specific measures to meet the strict environmental constraints, and correspondingly high are the costs accruing with such paper / paperboard container incineration.

A particularly important field of application of the pulp is hygienic products, and here diapers and incontinence products. These contain usually cellulose fibers, plastics (LDPE, PP, superabsorption polymer (SAP)), urine and excrements, and accrue especially in old people's homes, hospitals, and in private households with little children. Incontinence products—in the following referred to as ICP—meanwhile make up for approximately 15 to 20% of the entire waste appearance in Germany and have so far been collected as a mixed fraction via the household waste-like industrial waste or the household waste, respectively. Since the implementation of the Technical Instructions on Municipal Waste in June 2005, these ICP may no longer be deposited without being treated, but have to be incinerated in household waste incineration plants or in waste-fueled power stations. At present, a uniform concept for the collection or the disposal of ICP does not yet exist, and alternative possibilities of disposal are not available, either.

Some efforts of composting ICP and other pulp-containing composites such as, for instance, food packaging are indeed known—due to the high share of plastics foils in the rotten material, however, problems that could not be mastered arose, so that these efforts were cancelled. Substantially, the initially mentioned incineration in household waste incineration plants or in waste-fueled power stations presently remains as an alternative. Due to the comparatively low fuel value of these pulp-containing wastes and due to the volume of waste products provided for incineration which has risen abruptly owing to the Technical Instructions on Municipal Waste, there are presently not sufficient capacities available for the approved disposal of this notifiable waste. Due to this disposal bottleneck, the dumping grounds closed in June were opened again for a limited period of 12 months, so that the bottleneck has been removed for a short time—a medium-term or a long-term solution is not available. The situation is still aggravated by the fact that an EC standard for waste storage and waste treatment is pending to be adopted, pursuant to which only household waste that has been pretreated mechanically-biologically may be stored from 2008 on. This means, at the latest at this time the same mass problems that are presently existing in Germany due to the implementation of the Technical Instructions on Municipal Waste will exist all over Europe.

Due to the very high disposal prices of the communities which tend to be increasing further, more and more care facilities have been searching more cost-efficient concepts for their notifiable incontinence waste, wherein, as a rule, the manufacturers or deliverers of the ICP are addressed. The examples of automobile or electrical industries which take their products back at the end of the duration show a change in product responsibility, so that effective preparation concepts are searched by the manufacturers and the deliverers of the ICP, too.

In the domain www.knowaste.de, a recycling method for ICP is disclosed in which they are comminuted and subsequently divided into pulp, plastics materials, and deactivated EAP by means of a chemical treatment and a dividing stage.

The problem about this method is that the process is relatively complex due to the chemical treatment and should not suffice the demands of a mechanical-biological preparation as defined by the EC standards to be expected.

As compared to this, it is an object of the invention to provide a method and a fermentation plant by means of which pulp-containing waste, in particular ICP, can be prepared in a simple manner.

### SUMMARY OF THE INVENTION

This object is solved by the feature combination of claim 1 with respect to the method, and by the feature combination of claim 12 with respect to the fermentation plant.

In accordance with the invention, the pulp-containing waste is first of all mechanically prepared and, in so doing, comminuted. The mechanically prepared waste is then diluted (slurried) with process/press water, wherein some of the substances of content are already dissolved. In a subsequent method step, this suspension is, in a biological preparation step, disintegrated, sanitized, and organic components are methanized. The digestion residue remaining after the biological preparation step is divided into a plastics-rich and a pulp-rich fraction, and these fractions are then, in a conditioning stage, processed to recyclable material, or to fuel, or to products adapted to be deposited.

The method according to the invention and the fermentation plant according to the invention render it possible to generate, in an industrial scale, recyclable materials and fuel as well as biogas from the pulp-containing waste in an extremely simple and ecological manner.

In a preferred embodiment, the biological preparation stage comprises a sanitation at increased temperature and a subsequent methanization in a bioreactor (fermenter).

For accelerating the decomposition/disintegration of the biological components, it is possible to add nutrients for the microorganisms during the methanization or sanitation.

Due to the comparatively high plastics share, a lack of nitrogen exists during the biological preparation of the pulp-containing waste. To compensate for this lack of nitrogen, the invention suggests to add nitrogen-containing aggregates such as urea to the biological preparation.

The material flows in the method according to the invention and in the fermentation plant according to the invention may be controlled such that biologically active suspension components are withdrawn from one or several of the stages and are adapted to be fed back to one or several of the other stages or to the same stage at another place of the flow path as a vaccine or for adjusting a predetermined concentration profile. The withdrawn suspension portions may contain both swimming items and settling sediments. Furthermore, the water flows required in the process may be controlled between the individual stages in almost any manner so as to optimize the decomposition and cleaning processes.

The pulp-rich fraction is preferably conditioned to a low-caloric fuel and to other recyclable materials, the plastics-rich fraction to a high-caloric fuel and to recyclable materials such as plastics granulate.

The organic conversion and sanitation is improved if the suspension is impacted with shearing forces in the sanitation stage and during methanization.

The sanitation may be performed in several stages and may be put into practice in accordance with different concepts. In one concept, the sanitation is performed by means of gassing of one or several sanitation containers, so that, due to the hydrolization occurring by the impacting with process water and gas (air), the substrate is heated to the sanitation temperature (approx. 70° C.) and biological components are already disintegrated and dissolved. The sanitation containers may be connected hydraulically by means of an overflow.

Alternatively, the temperature increase may also be effected in that a suspension heated by means of a heat exchanger is supplied to the sanitation stage.

Basically, the sanitation stage may consist of a sanitation apparatus with two sanitation containers connected in series, each of them comprising a stirring device. It is preferred if the drain of the upstream sanitation container is connected with a heat exchanger, the output of which is adapted to be connected with an inflow of this sanitation container and/or of the downstream sanitation container via valve means.

Alternatively, the sanitation apparatus may also be designed as a horizontal container that is subdivided into a first and a second sanitation chamber by a separating wall provided with a passage. The suspension is in both chambers impacted with shearing forces by a joint stirring device, wherein the suspension is again, similar as in the afore-described embodiment, taken to sanitation temperature by means of a heat exchanger.

In a preferred embodiment of the invention, the sanitation and the methanization are performed in a compact reactor through which the suspension is, for instance as a plug flow, transported from a suspension inlet to a suspension outlet. The container is subdivided into a sanitation stage and a downstream methanization/fermentation stage by means of an intermediate wall with suspension passage. For improving the sanitation, the sanitation stage may again be subdivided into two chambers.

Since the dwell time in the fermentation stage is substantially longer than in the sanitation stage, the latter is designed with a minor axial length than the fermentation stage.

For the adjustment of a predetermined temperature profile inside the container, the container is isolated and is heated at least in sections.

The separation of the fermentation products is preferably performed in a separating container in which the pulp can be removed from the plastics by the applying of shearing forces, and in which, after the applying of the shearing forces, for instance, on switching off a stirring device, a layer with a plastics-containing swimming layer, a pulp-containing bottom layer, and an intermediate aqueous zone is formed, so that these fractions are easy to extract from the separating container.

The adjustment of the material flows to and between the individual stages (sanitation, methanization, separating facility) is, in accordance with the invention, performed via a dosing station that is connected with the stages mentioned via a suitable pipe installation, so that material flows can be extracted from one or several of the stages and be fed to one or several other stages or to the same stage in another place as a vaccine or for adjusting a predetermined concentration profile. The term “dosing station” means a pumping device with a pertinent pipe installation and valve arrangement which renders it possible to circulate and to convey material flows between or within the stages.

The supplying of the nutrients and of the other aggregates for improving the biological conversion may also be controlled via this dosing station.

For dissolving or suspending the mechanically prepared, pulp-containing waste, a pulper is preferably used.

Other advantageous further developments of the invention are the subject matter of further subclaims.

### BRIEF DESCRIPTION OF THE DRAWINGS

In the following, preferred embodiments of the invention are explained in more detail by means of schematic drawings. There show:

FIG. 1 method diagrams of methods according to the invention for the anaerobic treatment of pulp-containing wastes;

FIG. 2 plant diagrams of fermentation plants in accordance with the invention;

FIG. 3 a compact reactor for the sanitation and methanization of the pulp-containing wastes;

FIG. 4 an alternative solution with a separate sanitation apparatus and two bioreactors connected in parallel, which may be enlarged arbitrarily for adding further reactors;

FIG. 5 an embodiment similar to that of FIG. 3, wherein the sanitation apparatus is designed with two sanitation containers that are connected in series.

### DETAILED DESCRIPTION OF THE DRAWINGS

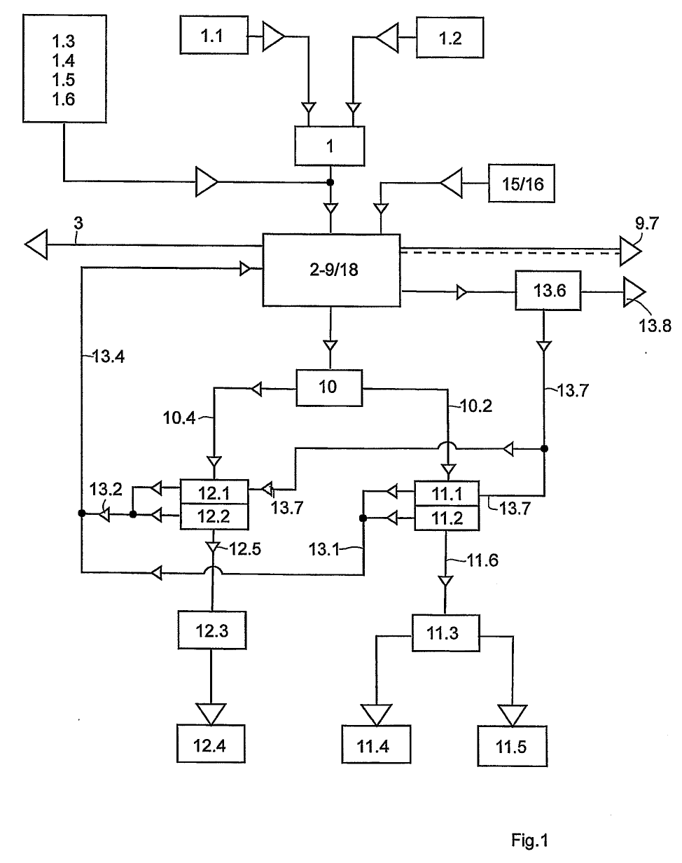

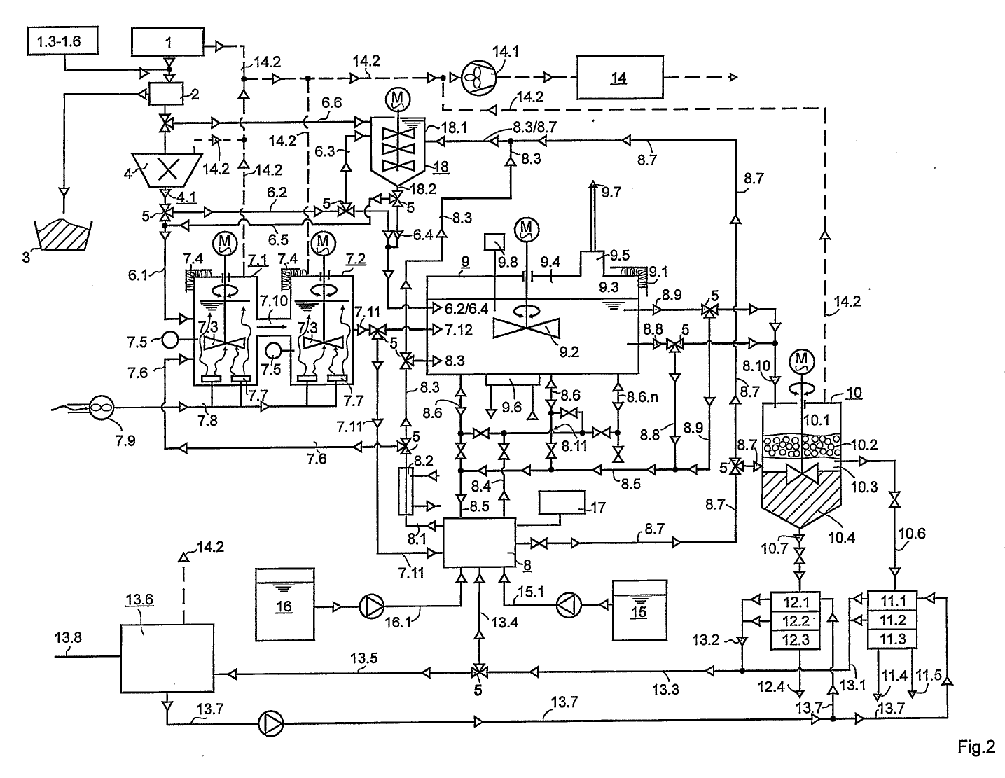

FIG. 1 illustrates the basic method diagram of methods in accordance with the invention for the anaerobic treatment of pulp-containing waste, such as paper, paperboard containers, composites, pulp waste, and ICP. The methods illustrated in the diagrams of FIGS. 1 and 2 substantially differ in that, on the one hand, ICP from old people's homes, hospitals, or households 1.1, 1.2, and, on the other hand, other pulp-containing wastes such as paper, paperboard containers, composites (for instances packages of food technology) (1.3 to 1.6) are processed. By means of FIG. 1, the preparation of ICP will first of all be described.

ICP (diapers) contain, as a rule, an absorbing center of fluffed pulp and superabsorption polymer (SAP), a permeable nonwoven layer on the upper side, and a barrier layer consisting of a polyethylene film. These layers are stuck with each other (polymer mixture). For fixing the ICP, adhesive strips are used which consist substantially of a polypropylene film. Elastic filaments that are arranged at the longitudinal side enable better adaptation to the body shape—these filaments consist, as a rule, of polyurethane. This means, such ICP consist substantially of pulp, of the SAP, and of a polymer mixture, in the following referred to as plastics. When the ICP are used, urine and excrements will be added, of course.

The concept according to the invention provides that these notifiable ICP are collected separately. Presently, hospitals and old people's homes are already committed to collect the accruing ICP separately. In some federal states, the separate collection of baby diapers is also mandatory (diaper sack). The ICP from hospitals and old people's homes 1.1 and the baby diapers 1.2 which are delivered separately are first of all collected in a bunker 1, and the sacks/bundles containing the ICP are opened as the case may be. The bunker 1 is equipped with dosing means via which the ICP can be fed to the further method stages. From the bunker 1, the ICP first of all arrive at a mechanical and biological preparation indicated by reference numbers 2 to 9, wherein the mechanical preparation (with respect to details see FIG. 2) comprises an interference and foreign material detection for the separation of foreign material and of noxious matter 3, a comminution device 4, dosing means 5 via which the comminuted ICP can either be fed to a sanitation or to a methanization, conveying means 6 for conveying these material flows to sanitation or to methanization, a central dosing station 8 for controlling the material flows fed to the individual method stages and extracted therefrom, and a bioreactor 9. In the following, the dosing means 5 refers to a slide or valve arrangement via which a material flow with an adjustable volume flow relation can be divided into at least two partial flows. For sanitation and methanization, press or process water 13.4 guided in the cycle is introduced, the loaded process water accruing after the mechanical-biological preparation is fed to a mechanical-biological waste water preparation 13.6, and possibly accruing surplus water 13.8 is drained. The cleaned process water is again fed into the process cycle as industrial water 13.7. As is indicated with 9.7 in FIG. 1, biogas 9.7 is generated by the methanization in the bioreactor, which may be supplied to an energetic utilization, for instance, in a heat plant.

The rotten material present after the methanization is supplied to a separating container and divided there into a plastics-rich fraction 10.2 and a pulp-rich fraction 10.4. The pulp-rich fraction 10.4 is fed to a pulp conditioning means 12 consisting of a dehydration means 12.1 and a dryer 12.2, and the dried fraction 12.5 that is then available is supplied to a conditioning stage 12.3 in which this fraction is preferably conditioned to a low-caloric fuel 12.4. The press water 13.2 accruing during dehydration and drying is fed to the process water cycle. In the embodiment shown, the dehydration means is designed with a washing device for cleaning the pulp. This dehydration may, for instance, be performed by the adding of industrial water 13.7 that is branched off after the waste water preparation.

The preparation of the plastics-rich fraction 12.0 is performed correspondingly. It is dehydrated in a dehydration press 11.1, wherein the cleaning of the plastics chips by means of industrial water 13.7 is first of all performed prior to the pressing process. In the subsequent step, the dehydrated plastics-rich fraction is dried in a drier. The press water 13.1 accruing during the dehydration and the washing process as well as the drying is added to the press water or waste water mixture 13.4 that is conveyed back to the mechanical-biological preparation stage.

The dried plastics-rich fraction 11.6 is then fed to a granulator 11.3 for preparation of the plastics material. This granulate may be sold directly as an industrial product 11.4, or else be pressed to a high-caloric fuel 11.5 (heating value greater than 20,000 kJ/kg). The process/press water cycle is illustrated in a strongly simplified manner in FIG. 1—with respect to the actual course of the water cycle, reference is made to FIG. 2.

The afore-describe method diagram reveals that no foreign energy has to be supplied, but that energy is generated by the biogas, and that high-value fuel or recyclable materials that can be utilized as industrial products accrue as method products. Pursuant to careful estimations, the costs for a preparation of ICP of presently 130.00 EURO per ton may be reduced to less than 80.00 EURO per ton with the afore-described method, so that this method constitutes a most interesting alternative also for the carriers of hospitals and old people's homes.

The preparation of other pulp-containing wastes may also be performed in the afore-described manner. In most cases, this pulp has to be diluted with process water after the mechanical comminution and prior to the sanitation or methanization. These pulp-containing wastes are predominantly wastes that are burdened with organic matter, which are sorted out in sorting facilities of plants for the treatment of residual waste (negative sorting). As pulp-containing wastes, paper 1.3, cardboard products 1.4, composites 1.5, and other pulp wastes 1.6 come into question. These pulp-containing wastes 1.3, 1.4, 1.5, 1.6 are, as a rule, solved in a pulper 18 after the mechanical preparation (detection of interference material and foreign material and possibly comminution), and prior to the biological preparation (sanitation and/or methanization). Such a pulper 18 is a large stirring container into which the waste material to be solved, on the one hand, and process water, on the other hand, are introduced. By means of intensive mixing by a stirring device, the soluble components of the waste are solved in process water and the portions of solid material are suspended. The dwell time in the pulper depends substantially on the solubility of the substances of content. In the case of large material flows, several pulpers 18 that are connected in parallel or in series may be operated.

As will be explained in detail by means of FIG. 2, the material flow extracted from the pulper may then be supplied to the sanitation apparatus 7 or to the bioreactor 9. Basically, it may be of advantage to also supply the ICP to a pulper 18 prior to the biological preparation so as to facilitate the biological preparation.

For the rest, the pretreatment of the pulp-containing wastes corresponds to the afore-described method for the preparation of ICP, so that these statements are to be applied correspondingly to pulp-containing wastes.

FIG. 2 illustrates a concrete plant diagram for the methods explained by means of FIG. 1. With such a plant, both ICP and pulp-containing waste (paper 1.3, cardboard products 1.4, composites 1.5, other pulp-containing waste 1.6, or the like) can be prepared in a pure fraction or as a mixed fraction. In accordance with FIG. 2, the ICP 1.1. and 1.2 incorporated in the bunker 1 and delivered separately from the other waste are conveyed to the detection of interference and foreign materials 2 via the dosing means, and the interference materials accruing are, via suitable sorting and separating devices, fed to an interference material container for disposal.

In the case in which the waste 1.1, 1.2, 1.3, 1.4, 1.5, or 1.6 is delivered in a form that is comminuted as far as possible, this material flow 6.6. that is available with solid matter shares with comparatively little size (10 to 30 cm² base area) may be supplied to the afore-mentioned pulper 18. As already stated, the soluble components of the material flow 6.6 are solved in process water 8.3/8.7 supplied to the pulper 18, wherein this process of solving is supported by mixing through by means of the stirring device. In this pulper, the material flow 6.6. is diluted to dry matter contents DM of approximately 5 to 15%. The material flow 18.2 extracted from the pulper 18 is either fed to the bioreactor 9 or to the sanitation apparatus 7 upstream thereof. The subdivision of the material flow 18.2 is performed through a dosing means 5 in which the material flow 18.2 is guided to the sanitation apparatus 7 (material flow 6.5) or to the bioreactor 9 (material flow 6.4). Basically, a respective partial flow to the apparatuses 9, 7 may also be branched off through the dosing means 5. In practice, it has turned out that, with the preparation of incontinence products, a dilution of the material flow in a pulper 18 is not necessary, so that the ICP may be fed directly to the comminution device 4.

The ICP freed from interference material and/or other pulp-containing wastes are then comminuted in a comminution means 4, so that they are, for instance, available in strips with a base area between 10 and 30 cm². The comminuted material flow 4.1 is, through a dosing means 5, either supplied to the pulper 18 or to the sanitation apparatus 7, or to the bioreactor 9. Exhaust air generated in the bunker 1 or in the comminution means 4 is sucked off via a blower 14.1 and is fed to an exhaust air cleaning facility 14 that comprises, for instance, a washing device and a biofilter for removing biological components and for the neutralization of smell.

In the case in which the pulp-containing waste 1.3, 1.4, 1.5, 1.6 is not available with the required dry matter contents, the comminuted waste 4.1 available after the comminution means 4 may be redirected as material flow 6.4 through the dosing means 5 and another dosing means 5 to the pulper 18 in which this material flow 6.3 is diluted to the desired dry matter contents (5 to 15%), and in which the soluble components in the material mixture 18.1 are solved. As already described, the material flow 18.2 extracted from the pulper is then supplied through another dosing means 5 either to the sanitation apparatus 7 (material flow 6.1), or to the bioreactor (material flow 6.4).

The material flow 6.1 adjusted via the dosing means 5 is introduced into the sanitation apparatus 7, for instance, overhead. In the embodiment illustrated in FIG. 2, the sanitation apparatus 7 comprises two sanitation containers 7.1, 72 that are connected with each other via a free overflow 7.10. In each of the sanitation containers 7.1, 7.2, a stirring device 7.3 is arranged via which shearing forces may be introduced into the suspension of comminuted waste 1.1, 1.2, 1.3, 1.4, 1.5, 1.6 and process or press water 7.6 supplied. The temperature in the sanitation apparatus 7 is monitored via a temperature check 7.5. For the aerobic heating of the suspension, air is supplied via ventilation elements 7.7 and an air distribution line 7.8, said air being sucked in via an air blower 7.9 and being conveyed to the ventilation elements 7.7. In the embodiment shown, this air blower 7.9 is illustrated as a rotary piston blower with a free intake manifold, but it is a matter of fact that other constructions may be used, too. In the case of waste that is difficult to prepare, a gas enriched with oxygen or pure oxygen may also be supplied instead of air.

By the gassing via the ventilation elements 7.7 and the press or process water 7.6 supplied, the suspension is sanitized since the temperature in the sanitation container 7.1, 7.2 increases due to the hydrolization (aerobic hydrolization) of the organic components. This temperature increase may be controlled by the amount of air supplied as a function of the signal of the temperature check 7.5. To reduce heat losses, the sanitation apparatus 7 is provided with an isolation 7.4. Via the stirring device 7.3, shearing forces are introduced into the suspension, so that it homogenizes inside the container and the mass transfer face is maximized, wherein a pre-separation into pulp and plastics components already takes place. The overflow 7.10 is adjusted such that the suspension, after a particular dwell time in the first sanitation container 7.1, flows over to the second sanitation container 7.2, and is further sanitized and hydrolized there. Also in this sanitation container 7.2, the air supply is performed as a function of the signal of the temperature check 7.5. The nitrogen-burdened exhaust air 14.2 accruing during hydrolysis is sucked off through the blower 14.1 and is cleaned in the exhaust air cleaning facility 14 containing an acid washing device.

After a dwell time of approximately one hour at 70° C., the suspension is, pursuant to the pertinent EU standards, unobjectionable with respect to hygiene and human medicine. However, to achieve a specific hydrolysis, the dwell time of the material mixture in the sanitation apparatus 7 should be at least two days. If only sanitation is desired, it may be performed in one single container 7.1 or 7.2, the content of which is—as will be explained in the following—circulated in the repumping method and heated through a heat exchanger.

The sanitized and—in the sanitation apparatus illustrated in FIG. 2—partially biologically disintegrated and hydrolized suspension is then extracted via a suction line 7.11 and, through another dosing means 5, either directly supplied as substrate 7.12 to the bioreactor 9, and/or to the central dosing station 8. Accordingly, the subdivision of the material flows extracted is performed by a suitable adjustment of the dosing means 5.

The substrate 7.12 is fed into the bioreactor 9 that is preferably designed as a horizontal container from the front side. In the bioreactor 9, a stirring device 9.2 is provided via which shearing forces may be applied into the substrate 7.12 introduced via the overflow. After the sanitation/hydrolization it is present with approximately 70° C. The methanization in the bioreactor 9 usually takes place in the thermopile range of approximately 55° C., so that heat is applied into the bioreactor through the substrate, and correspondingly less energy has to be supplied to compensate for radiation losses. This energy supply is, for instance, performed via an outer casing heating 9.6 of the bioreactor 9 that is provided with an isolation 9.1. In addition to the substrate 7.12 flowing over from the sanitation apparatus 7, the dosing station 8 conveys press or process water 8.1 to the bioreactor 9, and it is taken to its operating temperature in a heat exchanger 8.2. Downstream of the heat exchanger 8.2, further dosing means 5 is provided, via which the press/process water flow can be adjusted in the direction to the sanitation apparatus (process water flow 7.6) and in the direction to the bioreactor 9 (process water flow 8.3). The process water flow 8.3 may then be supplied to the bioreactor 9 and/or to the pulper 18 via further dosing means 5. As is further illustrated in FIG. 2, process water 8.7 that has not been pre-heated may be fed into the pulper 18 via the central dosing station 8 and a loading line 8.7 as well as further dosing means 5, so that a suitable temperature supporting the solving process can be adjusted in the pulper 18 by adjusting the mixture relation between the process water flows 8.3 and 8.7 to the pulper 18.

Via the process water flow 8.3 supplied to the bioreactor 9, the substrate in the bioreactor 9 is placed to a dry matter content that is optimal for the biological conversion. The temperature in the bioreactor 9 is detected via a further temperature check 9.8, the signal of which is used for controlling the dosing means 5 for adjusting the process water flow 8.3 and for adjusting the outer casing heating 9.6. Furthermore, the substrate volume flow 7.12 flowing over from the sanitation apparatus may also be adjusted as a function of the signal of the temperature check 9.8. In the bioreactor, the sanitized waste chips are, with a thermopile operation at approximately 55° C. and with the exclusion of air, subject to an anaerobic rotting process in which the components capable of decomposing, such as excrements and pulp, are biologically transformed and converted to biogas. This biogas that is present in the gas chamber 9.4 is extracted from the bioreactor 9 via a gas outlet tower 9.5 and is supplied to an energetic use.

The hydraulic dwell time of the material mixture 9.3 in the bioreactor 9 is approximately 18 days. The dry matter contents of the substrate mixture lies between 5 and 15% (between the third and fifth days). In the bioreactor 9, the pulp is separated from two-dimensional plastics parts due to the microbiological effect and the shearing forces applied. These plastics foils have a surface roughened up in the micro range, so that, with a square meter of plastics foil, a specific surface of approx. 50 m² is provided for bacterial population, and the organic conversion is performed correspondingly efficiently. The bacterial flora accumulates on this plastics surface, thus the active bacterial density is substantially increased vis-á-vis a “smooth” substrate with a low specific surface. Furthermore, it is of advantage that these immobilized microorganisms verifiably develop higher activities than freely movable organisms. The efficient mass transfer face is optimal with the afore-mentioned face (10 to 30 cm²) of the comminuted plastics chips.

By means of the stirring device 9.1, the entire suspension 9.3 is recirculated and thus its concentration is rendered uniform, and the development of swimming covers is largely avoided.

A particularity of the plant diagram illustrated in FIG. 2 consists in that the bioreactor 9 is designed with a plurality of recirculation and cycle connections 8.6 to 8.6n that are connected with the central dosing station 8 via a cycle/recirculation pipe installation (with the required valve elements) as well as an inflow 8.4 and a drain 8.6, so that suspension/substrate 9.3 may be extracted via one or a plurality of the recirculation and cycle connections 8 and may be supplied again via other ones of these recirculation and cycle connections. Thus, it is possible to perform a partial mixing through and remixing between the inlet side (at the left in FIG. 2) and the drain side (at the right in FIG. 2) within the reactor alone by these recirculated material flows. The dosing station is further connected with a urea container 16 via dosing means indicated with 16.1, and with a nutrient solution container 15 via further dosing means 15.1, so that, via the dosing station 8, urea, nutrients, or the like may be fed into the bioreactor 9, the pulper 18, or also to the sanitation apparatus 7 or the separating container 10. By means of the stirring device 9.2, the suspension 9.3 is moved, and the nutrients and urea that are, for instance, supplied via the pipe installation 8.11, are constantly fed to the immobilized bacteria that will then generate methane gas, carbonic acid, and traces of hydrogen sulfide as a metabolite.

The adding of urea is of advantage since the nutrient relation of used incontinence products is not balanced. On average, the CSB/N relation is about 170:1. For carbon-rich substrates this relation in the bioreactor should, however, range at approximately 60:1. Thus, a lack of nitrogen exists. To compensate for this lack of nitrogen, nitrogen, e.g. in the form of urea, is added to the process in a dosed manner. Basically, it is also possible to supply further aggregates such as, for instance, clearing sludge, through which the fermentation may be stabilized and improved, via the dosing station 8. Since, however, by the addition of clearing sludge the fermented, almost authentic fraction is additionally polluted, the addition of selected nutrients is preferred for supporting fermentation in accordance with the invention. The ph value that is optimal for the biological conversion may be adjusted via a ph value control 17 and the dosing means 8. Since the dosing means may be operated both in the suction and in the pressure operation, the biologically active vaccine mass such as, for instance, plastics particles populated by microorganisms, may be transported in zones in which bioactivity takes place in a reduced manner, or in which the ph value has to be adjusted. Basically, the nutrients and the aggregates for adjusting this nitrogen content may also be supplied directly, i.e. irrespective of the dosing station 8.

For stabilizing and possibly even controlling the metabolic process, the stirring device 9.2 is stopped before the fermented residue is discharged. Subsequently, the rising plastics-rich fraction immediately separates from the sinking pulp-rich fraction. These fractions of the fermented residue (rotted material) are extracted through drain lines 8.9 (top) and 8.8 (bottom) designed as overflow lines, and guided to the separating container 10 through the joint overflow line 8.10 and fed into it overhead. This separating container is also designed as a standing container and equipped with a stirring device 10.1 for mixing through the rotted material. It is also possible to supply both fractions directly to the separating container 10 via a joint overflow with the stirring device in operation (not illustrated). The drain lines 8.9 and 8.8 are, via the recirculation/cycle pipe installation 8.11, connected with the dosing station 8 and thus with the recirculation and cycle connections 8.6, so that it is also possible to specifically supply back pulp-rich or plastics-rich fermented residue with corresponding microorganism population into the bioreactor, and/or to feed them in as vaccine in another stage of the plant. Via the dosing station 8, it is also possible to directly convey the substrate from the sanitation apparatus 7 or—preferably—from the bioreactor 9, or process water to the separating facility 10. This material flow is designated with 8.7 in the illustration according to FIG. 2.

The separating container 10 according to FIG. 2 is operated in stages, wherein first of all the rotten material introduced into the separating container 10 via the overflow line 8.10 is mixed through, and the pulp fibers and the bio film are removed from the plastics components by means of the shearing forces applied.

On standstill of the stirring device 10.1, the swimming plastics-rich fraction 10.2 separates within few minutes from the sinking pulp-rich fraction 10.4, wherein an aqueous zone 10.3 (cloudy water) is formed between the two layers. The plastics-rich fraction 10.2 swimming on the cloudy water 10.3 is extracted via a suction line 10.6, and the sinking pulp-rich fraction 10.4 is extracted via a suction line 10.7. FIG. 2 does not illustrate the possibility of returning the foil chips (10.2) accruing in the separating container 10 and populated with microorganisms again to the bioreactor 9 via a return device (for instance, by connection with the dosing station 8).

The plastics-rich fraction 10.2 is fed to a plastics conditioning means 11 that consists substantially of the dehydration press 11.1, the dryer 11.2, and a granulator 11.3. The dehydration press 11.1 is provided with a washing device in which the plastics chips of the plastics-rich fraction 10.2 can be cleaned by the addition of industrial water 13.7. The dryer 11.2 comprises a condenser for drying the plastics chips available after the dehydration press. In the granulator 11.3, the dried plastics are finally granulated and possibly pressed to the high-caloric fuel 11.5. The plastics granulate 11.4 accruing may be sold directly. The press water 13.2 accruing during dehydration and drying is, via further dosing means 5, either supplied to the dosing station 8 as process water mixture 13.4, and conveyed therefrom to the afore-described stations 7, 9, 10, or 18, so that the press water is substantially guided as cycle water. A portion 13.5 is transmitted from the dosing means 5 to a mechanical-biological waste water preparation 13.6 for denitrification and sanitation and for the preparation of industrial water.

The exhaust air 14.2 accruing during waste water preparation is fed to the exhaust air cleaning facility 14. The denitrified surplus water 12.8 is transferred to the municipal sewage plant. The majority of the denitrified waste water is returned to the two conditioning facilities 11, 12 as industrial water 13.7.

The pulp-rich fraction 10.4 is—as mentioned—conveyed via the suction line 10.7 to the pulp conditioning means 12 and there—like in the plastics conditioning means 11—dried by means of dehydration means with integrated washing device 12.1. and a dryer 12.2, and, via a conditioning stage 12.3, prepared to a regular fuel with an adjusted fuel value between 3000 to 5000 kJ/kg. In so doing, pelletizing may be performed, so that this low-caloric fuel is adapted to be supplied to a wood chip device. The process water 13.7 is, correspondingly as with the preparation of the plastics-rich fraction, added to the washing device of the dehydration means 12.1, and the press water 13.2 accruing after the dehydration means and the dryer is mixed to the waste water mixture 13.3 that is then prepared in the waste water preparation facility 13.6.

In the afore-described embodiment, two water cycles exist at first sight. On the one hand, the process/press water for adjusting the dry matter contents in the sanitation, the pulper 18, the methanization, and the drying facility, and, on the other hand, the industrial water cycle for conditioning the plastics-rich and the pulp-rich fraction. The two cycles are, however, connected with each other via the dosing means 8, so that it is possible to correspondingly feed volume flows from the one cycle into the other cycle.

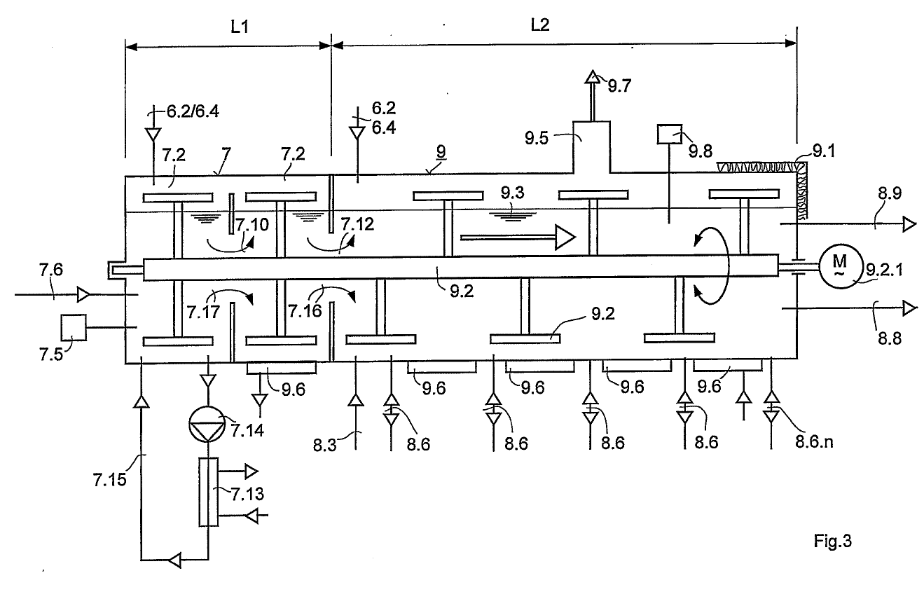

In the afore-described embodiment, the sanitation apparatus 7 and the bioreactor 9 are designed separately, wherein the sanitation apparatus 7 on its part consists of two containers 7.1, 7.2. The technical effort with respect to the apparatus when putting such a solution into practice is relatively high. FIG. 3 shows an embodiment in which the sanitation and the fermentation are performed in one single compact reactor. It is designed as a horizontal container and is provided with an isolation 9.1 so as to avoid heat losses. The comminuted waste chips available after the comminution means 4 are fed as material flow 6.1. or 6.5 (from pulper 18) at the left end section of the container, and correspondingly the heated process water 7.6 available after the heat exchanger 8.2 is fed into the container from the front side. The plastics-rich fraction and the pulp-rich fraction of the fermented residue are extracted at the front side at the right end section of the container via the drain lines 8.9 or 8.8, so that a plug flow from the left to the right (view of FIG. 3) results inside the container. This plug flow is supported by a stirring device 9.2 that is positioned in the container. The container is subdivided into a sanitation stage with the axial length L1 and a methanization stage with the axial length L2 by an intermediate wall 7.16, wherein the axial length L2 is substantially greater than L1. Thus, it is taken into consideration that the hydraulic dwell time for methanization is substantially larger than for sanitation. The material flow 6.2 or 6.4 may also be fed directly to the methanization.

The chamber with the length L1 constituting the sanitation stage 7 is on its part subdivided into two chambers 7.1, 7.2 through an intermediate wall 7.17, wherein stirring organs of the stirring device 9.2 are positioned in every chamber 7.1, 7.2. The temperature in the chamber 7.1 of the sanitation stage 7 may be collected via the temperature check 7.5, and the temperature in the chamber forming the bioreactor 9 may be collected by a temperature check 9.8. The chamber 7.1 comprises a drain connection from which suspension can be extracted via a circulation pump 7.14 and can be taken to the sanitation temperature in a heat exchanger 7.13. The heated suspension is then again returned to the chamber 7.1 via a repumping line 7.15 and an inflow. For adjusting the sanitation temperature and the optimal temperature for methanization, an outer casing heating 9.6 comprising heating segments that are adapted to be controlled separately from each other is further designed. The separating wall 7.17 and the intermediate wall are designed with passages or overflows, so that the suspension to be prepared is movable as plug flow from the left to the right in the container. The biogas accruing during methanization is extracted via the gas outlet tower 9.5 formed at the container portion 9.

As in the afore-described embodiment, it is possible to extract and to feed material flows from the portion forming the bioreactor 9 via the dosing means 8 and the recirculation and cycle connections 8.6 to 8.6n so as to generate remixtures and cycle mixtures, and to thus adjust a predetermined concentration and temperature profile in the reactor, and/or to feed the afore-described aggregates.

The reactor according to FIG. 3 is characterized by an extremely compact geometry, wherein the heat losses and the pipe installation effort are reduced to a minimum due to the short distances between the individual stages (sanitation, methanization). Another advantage consists in that only one single stirring device with one single stirring device drive 9.2.1 is required for both stages, so that the effort with respect to device technology is further minimized vis-á-vis the initially described solution. The stirring device drive 9.2.1 is designed reversible in all afore-described embodiments so as to apply different shearing forces and to reverse the transport means within the respective stage at short notice.

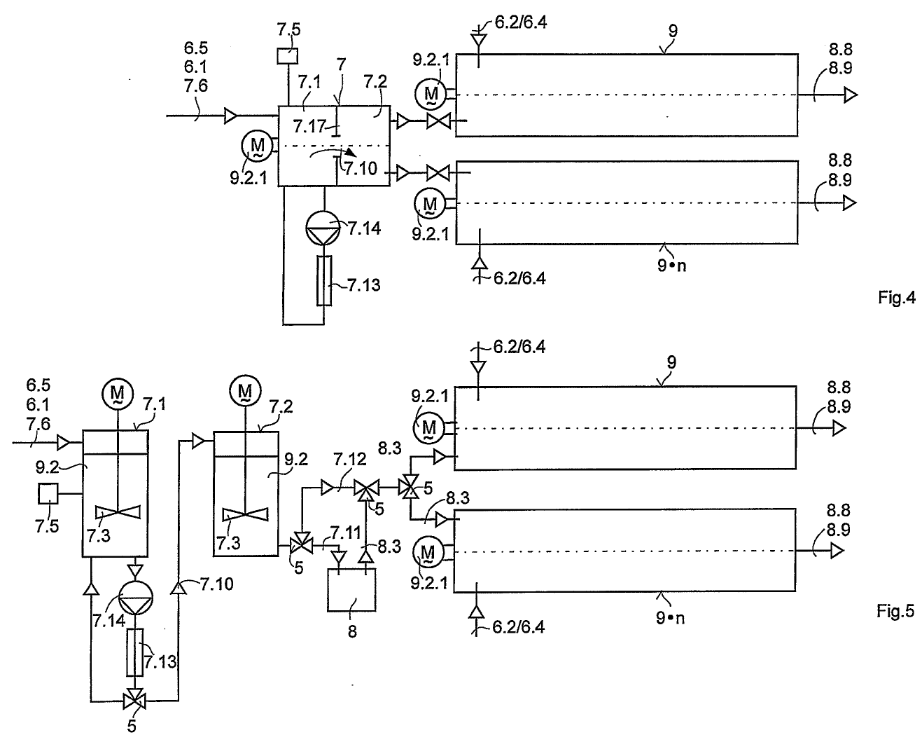

FIGS. 4 and 5 show further embodiments of the sanitation stage and the methanization stage, wherein both stages are put into practice by separate apparatuses.

FIG. 4 shows an embodiment in which the sanitation apparatus 7 is formed by one single container that is divided into a sanitation chamber 7.1 and a second sanitation chamber 7.2 by the separating wall 7.17. The separating wall 7.17 is designed such that an overflow 7.10 from the chamber 7.1 into the chamber 7.2 is possible. Similar as with the embodiment explained by means of FIG. 3, a drain is provided in the region of the first chamber 7.1, via which substrate may be extracted by means of the circulation pump 7.14 and be heated to sanitation temperature in the heat exchanger 7.13, and then be returned to the chamber 7.1 via the repumping line 7.15. As in the afore-described embodiments, the material flows 6.1, 6.5, 7.6 are fed into the chamber 7.1 at the front side, and the sanitation temperature is monitored by the temperature check 7.8. The application of shearing forces is performed via a joint stirring device with one single stirring device drive 9.2.1. The sanitized material flows (substrate 7.12) are extracted via two parallel drain lines 7.18 in this embodiment. Each of these drain lines 7.18 opens at the front side in a respective horizontal bioreactor 9, 9n that is designed in correspondence with the methanization stage 9 in FIG. 3, and thus comprises one stirring device each with a horizontal stirring axis. In each of the bioreactors 9, the substrate is conveyed through the stirring device 9.2 similar to a plug flow from the left to the right, and is then extracted at the front side as a plastics-rich or pulp-rich fermented residue. Basically, it is also possible to connect more than two bioreactors 9 in parallel.

FIG. 5 finally shows an embodiment in which, as in the afore-described embodiment, also several bioreactors 9 that are connected in parallel are used. The construction of the sanitation apparatus is, however, selected somewhat different—in the instant embodiment the sanitation apparatus 7 is designed by two sanitation containers 7.1, 7.2 that are arranged separately from each other, each of which is allotted with a stirring device 9.2. In FIG. 5, these containers 7.1, 7.2 are arranged upright—basically, it is, of course, possible to also use horizontal containers with a horizontal stirring device (see FIGS. 3, 4). The corresponding also applies, of course, for the embodiment of FIG. 2. In the embodiment illustrated, the stirring devices 7.3 are designed with paddle agitators, but other stirring organs may, of course, also be used. The temperature is collected via a temperature check 7.5, and the material flows 6.1, 6.5, 6.7 to be sanitized are fed into the first sanitation container 7.1 overhead. The first sanitation container 7.1 is again provided with a drain via which the suspension is adapted to be extracted by means of a circulation pump 7.14 and to be heated to sanitation temperature (approx. 70° C.) in the heat exchanger 7.13. The heated material flow is then again returned to the sanitation container 7.1 via the repumping line 7.15 or fed into the second sanitation container 7.2 as material flow 7.10 via dosing means 5, and the sanitized ICP substrate is, via a drain and suction line 7.11 and the dosing station 8, or directly (substrate material flow 7.12 indicated in dashes) conveyed to the methanization, wherein in the instant embodiment the subdivision of the material flows to the individual bioreactors 9 is performed via further dosing means 5.

Also in these embodiments (FIGS. 4, 5) the material flows 6.2 (after comminution) and 6.4 (after pulper 18) may be introduced into the bioreactor 9 directly.

The applicant reserves the right to direct independent claims to the different sanitation apparatuses 7, to the bioreactor 9, and to the separating facility 10 as well as the dosing station 8.

With the method according to the invention it is possible to convert the pulp almost completely to biogas, and to convert the plastics fraction to naphtha (diesel fuel, gasoline) in a plastics converting plant.

Disclosed are a method for the anaerobic treatment of pulp-containing wastes and a fermentation plant for such products, wherein they are first of all mechanically prepared and comminuted, subsequently diluted to a predetermined dry matter content by the addition of process water or the like, and sanitized and anaerobically fermented in a subsequent method step. The remaining rotted residue is divided into a plastics-rich and a pulp-rich fraction, and these fractions are conditioned in conditioning stages to recyclable materials, or to fuel, or to products adapted to be deposited.

Although the best mode contemplated by the inventors of carrying out the present invention is disclosed above, practice of the above invention is not limited thereto. It will be manifest that various additions, modifications and rearrangements of the features of the present invention may be made without deviating from the spirit and the scope of the underlying inventive concept.

1. A method for the anaerobic treatment of pulp-containing wastes, comprising the steps of:
mechanical preparation, in particular comminution of the wastes;
suspending/solving the comminuted wastes in process water;
disintegrating, sanitizing, and/or methanizing organic components of the suspension in a biological preparation stage;
dividing the remaining rotted residue into a plastics-rich and a pulp-rich fraction; and
conditioning the fractions to recyclable materials, or to fuel, or to products adapted to be deposited.

2. The method according to claim 1, wherein the biological preparation stage comprises sanitation at increased temperature and methanization in a bioreactor.

3. The method according to claim 2, wherein nutrients for accelerating the biological conversion are fed to the biological preparation stage, in particular the bioreactor.

4. The method according to claim 2, wherein nitrogen-containing aggregates are fed to the biological preparation stage.

5. The method according to claim 1, wherein the wastes are diluted to a dry matter content of 5 to 20%.

6. The method according to claim 1, wherein material flows are adapted to be extracted with a controllable amount from at least one of the stages, and to be supplied to one or a plurality of the other stages, or to the same stage at another place of the suspension flow path as a vaccine or for adjusting a predetermined concentration profile.

7. The method according to claim 6, wherein the extracted suspension portions contain swimming items or settling sediments.

8. The method according to claim 1, wherein the pulp-rich fraction is conditioned to a low-caloric fuel and to other recyclable materials, and the plastics-rich fraction to a high-caloric fuel and to recyclable materials such as plastics granulate.

9. The method according to claim 1, wherein the suspension is impacted with shearing forces in the sanitation stage and in the bioreactor.

10. The method according to claim 1, wherein the sanitation is performed in several stages.

11. The method according to claim 1, wherein the suspending, solving of the pulp-containing waste is performed in a pulper.

12. A fermentation plant for pulp-containing wastes, in particular for performing the method according to any of the preceding claims, comprising a mechanical preparation stage for comminuting the pulp-containing waste, a biological preparation stage in which the wastes are suspended in process water and biological components are methanized and discharged, a separating facility for dividing the rotted residue available after the biological preparation into a plastics-rich and a pulp-rich fraction, and a conditioning stage for preparing the respective fractions to at least one of a group including a fuel, a recyclable material, and a product adapted to be deposited.

13. The plant according to claim 12, comprising a gassed sanitation apparatus upstream of the methanization, said sanitation apparatus comprising at least two sanitation containers that are each designed with a stirring device and that are hydraulically connected with each other.

14. The plant according to claim 12, comprising a sanitation apparatus upstream of the methanization and designed with a stirring device, which a suspension heated to sanitation temperature is fed to.

15. The plant according to claim 14, wherein the sanitation apparatus comprises two sanitation containers connected in series, which are each designed with a stirring device, wherein the drain from the upstream sanitation container is connected with a heat exchanger, the output of which is, via dosing means, adapted to be connected with an inflow of the first container and/or an inflow of the downstream sanitation container.

16. The plant according to claim 14, wherein said sanitation apparatus is a horizontal container that is subdivided into a first and a second sanitation chamber by an intermediate wall with a passage, and which a joint stirring device is assigned to, wherein a drain from said first sanitation chamber is connected with a heat exchanger, the output of which is connected with an inflow of said first sanitation chamber.

17. The plant according to claim 14, wherein the sanitation and the methanization are performed in a compact reactor that is designed as a horizontal container through which the suspension is adapted to be conveyed via a stirring device, for instance, as plug flow from an inlet to an outlet, wherein the container is subdivided into a sanitation stage and a downstream fermentation stage through an intermediate wall with a suspension passage.

18. The plant according to claim 17, wherein said fermentation stage has a greater axial length (L2) than said sanitation stage.

19. The plant according to claim 17, wherein the container is provided with an isolation and is heated with a heating at least in sections.

20. The plant according to any of claim 12, wherein the methanization is performed in one or a plurality of bioreactors that are connected in parallel.

21. The plant according to claim 12, wherein the separating facility downstream of the biological preparation comprises a separating container in which the pulp can be removed from the plastics by the application of shearing forces, and in which, after the application of the shearing forces, a layer with a swimming layer of a plastics-containing fraction, a sinking layer of a pulp-containing fraction, and an aqueous zone therebetween is formed.

22. The plant according to claim 12, comprising a dosing station that is connected to the sanitation apparatus, the bioreactor, a pulper, and/or the separating container such that material flows are adapted to be extracted from one or a plurality of the stages and to be fed to one or a plurality of the other stages, or to the same stage in another place of the suspension flow path as a vaccine or for adjusting a predetermined concentration profile.

23. The plant according to claim 12, comprising a nutrient and an aggregate container from which nutrients for microorganisms, urea, or other aggregates can be added to the individual stages in a dosed manner so as to support the biological conversion.

24. The plant according to claim 12, comprising a pulper for solving/suspending the pulp-containing waste in process water.

25. The method according to claim 4, wherein the nitrogen-containing aggregates include urea.

26. The method according to claim 5, wherein the wastes are diluted to a dry matter content of 8 to 12%.
